(19) United States
(12) Patent Application Publication
(10) Pub. No.: US 2022/0362489 A1
(43) Pub. Date: Nov. 17, 2022

(54) PRESSURE BASED TEMPERATURE CONTROL OF A VAPORIZER DEVICE

(71) Applicant: JUUL Labs, Inc., San Francisco, CA (US)

(72) Inventor: Matthew Czapar, San Francisco, CA (US)

(21) Appl. No.: 17/766,068

(22) PCT Filed: Oct. 1, 2020
(86) PCT No.: PCT/US2020/053808

§ 371 Date: Apr. 1, 2022

Related U.S. Application Data
(60) Provisional application No. 62/910,742, filed on Oct. 4, 2019.

(51) Int. Cl. A61M 11/04

(52) U.S. Cl. CPC A61M 11/042; A61M 2205/3368; A61M 2205/3358; A61M 2205/121; A61M 2205/14; A61M 2205/3569; A61M 2205/50

(57) ABSTRACT

A vaporizer device (100) may be configured to adjust, based on ambient pressure, a setpoint temperature and/or a ramp rate of the vaporizer device. The setpoint temperature of the vaporizer device may be adjusted based on the ambient pressure in order to account for differences in the boiling point of a vaporizable material at different ambient pressures. Adjusting the setpoint temperature of the vaporizer device based on ambient pressure may further compensate for changes in the sensitivity of a user's taste buds in different ambient pressure and/or humidity environments. The ramp rate of the vaporizer device may be adjusted based on the ambient pressure such that the vaporizer device is able to achieve the higher boiling point of the vaporizable material in a higher ambient pressure environment in a same quantity of time as a lower boiling point of the vaporizable material in a lower ambient pressure environment.

### CROSS-REFERENCE TO RELATED APPLICATIONS

This application claims priority to U.S. Provisional Patent Application No. 62/910,742, filed on Oct. 4, 2019, the contents of which are herein incorporated by reference in its entirety.

### TECHNICAL FIELD

The current subject matter described herein relates generally to vaporizer devices, such as portable, personal vaporizer devices for generating and delivering an inhalable aerosol from one or more vaporizable materials, and more particularly relates to temperature control for vaporizer devices.

### BACKGROUND

Vaporizing devices, including electronic vaporizers or e-vaporizer devices, allow the delivery of vapor and aerosol containing one or more active ingredients by inhalation of the vapor and aerosol. Electronic vaporizer devices are gaining increasing popularity both for prescriptive medical use, in delivering medicaments, and for consumption of nicotine, tobacco, other liquid-based substances, and other plant-based smokeable materials, such as cannabis, including solid (e.g., loose-leaf or flower) materials, solid/liquid (e.g., suspensions, liquid-coated) materials, wax extracts, and prefilled pods (cartridges, wrapped containers, etc.) of such materials. Electronic vaporizer devices in particular may be portable, self-contained, and convenient for use.

### SUMMARY

Aspects of the current subject matter relate to temperature controls for a vaporizer device. In particular, in accordance with implementations of the current subject matter, the temperature of the vaporizer device, including a setpoint temperature of the vaporizer device and/or a ramp rate for adjusting the temperature of the vaporizer device, may be determined based at least on an ambient pressure.

According to an aspect of the current subject matter, a vaporizer device includes a pressure sensor configured to measure an ambient pressure, and a controller configured to: detect a change in the ambient pressure from a first ambient pressure to a second ambient pressure, the first ambient pressure associated with a first temperature at which a vaporizable material undergoes a phase transition, and the second ambient pressure associated with a second temperature at which the vaporizable material undergoes the phase transition; and in response to detecting the change in the ambient pressure, adjust a setpoint temperature of the vaporizer device, the setpoint temperature being adjusted based at least on the second temperature at which the vaporizable material undergoes the phase transition at the second ambient pressure, and the vaporizer device operating at the setpoint temperature in order to cause a vaporization of the vaporizable material.

According to an inter-related aspect, a method includes detecting, at a vaporizer device, a change in an ambient pressure from a first ambient pressure to a second ambient pressure, the first ambient pressure associated with a first temperature at which a vaporizable material undergoes a phase transition, and the second ambient pressure associated with a second temperature at which the vaporizable material undergoes the phase transition; and in response to detecting the change in the ambient pressure, adjusting a setpoint temperature of the vaporizer device, the setpoint temperature being adjusted based at least on the second temperature at which the vaporizable material undergoes the phase transition at the second ambient pressure, and the vaporizer device operating at the setpoint temperature in order to cause a vaporization of the vaporizable material.

According to an inter-related aspect, a non-transitory computer readable medium is provided, the non-transitory computer readable medium storing instructions, which when executed by at least one data processor, result in operations including detecting, at a vaporizer device, a change in an ambient pressure from a first ambient pressure to a second ambient pressure, the first ambient pressure associated with a first temperature at which a vaporizable material undergoes a phase transition, and the second ambient pressure associated with a second temperature at which the vaporizable material undergoes the phase transition; and in response to detecting the change in the ambient pressure, adjusting a setpoint temperature of the vaporizer device, the setpoint temperature being adjusted based at least on the second temperature at which the vaporizable material undergoes the phase transition at the second ambient pressure, and the vaporizer device operating at the setpoint temperature in order to cause a vaporization of the vaporizable material.

According to an inter-related aspect, an apparatus includes means for measuring an ambient pressure; means for detecting a change in an ambient pressure from a first ambient pressure to a second ambient pressure, the first ambient pressure associated with a first temperature at which a vaporizable material undergoes a phase transition, and the second ambient pressure associated with a second temperature at which the vaporizable material undergoes the phase transition; and means for responding to the change in the ambient pressure by adjusting a setpoint temperature of the apparatus, the setpoint temperature being adjusted based at least on the second temperature at which the vaporizable material undergoes the phase transition at the second ambient pressure, and the apparatus operating at the setpoint temperature in order to cause a vaporization of the vaporizable material.

In some variations, one or more of the features disclosed herein including the following features can optionally be included in any feasible combination. In response to detecting the change in the ambient pressure, a ramp rate at which a temperature of a heater of the vaporizer device is changed in order to achieve the setpoint temperature may be adjusted. A proportional-integral-derivative control may be applied to achieve the setpoint temperature, and the ramp rate may be adjusted by adjusting at least one of a proportional term, an integral term, or a derivative term of the proportional-integral-derivative control. The ramp rate of the vaporizer device may be adjusted such that the vaporizer device achieves the second temperature in a substantially same amount of time as the vaporizer device achieves the first temperature. The setpoint temperature of the vaporizer device may be adjusted to be equal to the second temperature. The setpoint temperature of the vaporizer device may be adjusted, based at least on a user preference for a strength of a vapor generated by the vaporization of the vaporizable material, to exceed the second temperature. The setpoint temperature of the vaporizer device may be further adjusted based at least on a type of the vaporizable material. The adjustment of the setpoint temperature of the vaporizer device may include adjusting a preset setpoint temperature of the vaporizer device, where the preset setpoint temperature includes a default setpoint temperature, a user defined setpoint temperature, and/or a previous setpoint temperature for the vaporizer device. In response to detecting the change in the ambient pressure, a notification may be sent to a user device coupled with the vaporizer device. The notification may indicates at least one of the change in the ambient pressure or the adjustment of the setpoint temperature of the vaporizer device. The notification may prompt a user to consent to the adjustment to the setpoint temperature of the vaporizer device, where the setpoint temperature of the vaporizer device is adjusted in response to the user consenting to the adjustment to the setpoint temperature of the vaporizer device. The vaporizer device may include a vaporizer body, the vaporizer body including a cartridge receptacle configured to receive a cartridge containing the vaporizable material, the cartridge further including a heater configured to deliver heat to the vaporizable material contained in the cartridge, and the heat delivered to the vaporizable material causing the vaporization of the vaporizable material. An insertion of the cartridge into the cartridge receptacle may be detected; and in response to the insertion of the cartridge, the pressure sensor may be activated to measure the ambient pressure. The vaporizer device may include a mouthpiece configured to enable a vapor to be drawn from the vaporizer device, the vapor being generated by the vaporization of the vaporizable material.

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A-FIG. 1F illustrate features of a vaporizer device including a vaporizer body and a cartridge, in accordance with some implementations;

FIG. 2 is a schematic block diagram illustrating features of a vaporizer device having a cartridge and a vaporizer body, in accordance with some implementations;

FIG. 3 illustrates communication between a vaporizer device, a user device, and a server, in accordance with some implementations;

FIG. 4 depicts a block diagram illustrating an example of proportional-integral-derivative (PID) control, in accordance with some implementations; and

FIG. 5 depicts a chart illustrating a process for adjusting the setpoint temperature and/or ramp rate of a vaporizer device, in accordance with some implementations.

When practical, similar reference numbers denote similar structures, features, or elements.

### DETAILED DESCRIPTION

The setpoint temperature of a vaporizer device may refer to the temperature at which a vaporizer device operates to vaporize a vaporizable material. Varying the setpoint temperature of the vaporizer device may alter the density of the resulting vapor and thus the flavor profile of the vapor experienced by a user. For example, a higher setpoint temperature may produce a denser vapor with a more concentrated flavor while a lower setpoint temperature may produce a less dense vapor with a less concentrated flavor. Ambient pressure around the vaporizer device, which may correspond to an atmospheric pressure, may determine the temperature at which the vaporizable material undergoes a phase transition (e.g., from a liquid to a vapor). Moreover, the flavor profile of the vapor experienced by the user may also change due to fluctuations in ambient pressure and humidity. Accordingly, the setpoint temperature and the corresponding ramp rate for maintaining a consistently optimal user experience may depend on the ambient pressure around the vaporizer device. As such, aspects of the current subject matter relate to controlling the temperature of the vaporizer device by at least adjusting, based at least on the ambient pressure, the setpoint temperature of the vaporizer device and the ramp rate for achieving the setpoint temperature of the vaporizer device.

Before providing additional details regarding aspects of temperature adjustment of a vaporizer device, the following provides a description of some examples of vaporizer devices including a vaporizer body and a cartridge. The following descriptions are meant to be exemplary, and aspects related to temperature adjustment consistent with the current subject matter are not limited to the example vaporizer devices described herein.

Implementations of the current subject matter include devices relating to vaporizing of one or more materials for inhalation by a user. The term “vaporizer” may be used generically in the following description and may refer to a vaporizer device, such as an electronic vaporizer. Vaporizers consistent with the current subject matter may be referred to by various terms such as inhalable aerosol devices, aerosolizers, vaporization devices, electronic vaping devices, electronic vaporizers, vape pens, etc. Examples of vaporizers consistent with implementations of the current subject matter include electronic vaporizers, electronic cigarettes, e-cigarettes, or the like. In general, such vaporizers are often portable, hand-held devices that heat a vaporizable material to provide an inhalable dose of the material. The vaporizer may include a heater configured to heat a vaporizable material which results in the production of one or more gas-phase components of the vaporizable material. A vaporizable material may include liquid and/or oil-type plant materials, or a semi-solid like a wax, or plant material such as leaves or flowers, either raw or processed. The gas-phase components of the vaporizable material may condense after being vaporized such that an aerosol is formed in a flowing air stream that is deliverable for inhalation by a user. The vaporizers may, in some implementations of the current subject matter, be particularly adapted for use with an oil-based vaporizable material, such as cannabis-derived oils although other types of vaporizable materials may be used as well.

One or more features of the current subject matter, including one or more of a cartridge (also referred to as a vaporizer cartridge or pod) and a reusable vaporizer device body (also referred to as a vaporizer device base, a body, a vaporizer body, or a base), may be employed with a suitable vaporizable material (where suitable refers in this context to being usable with a device whose properties, settings, etc. are configured or configurable to be compatible for use with the vaporizable material). The vaporizable material may include one or more liquids, such as oils, extracts, aqueous or other solutions, etc., of one or more substances that may be desirably provided in the form of an inhalable aerosol. The cartridge may be inserted into the vaporizer body, and then the vaporizable material heated which results in the inhalable aerosol.

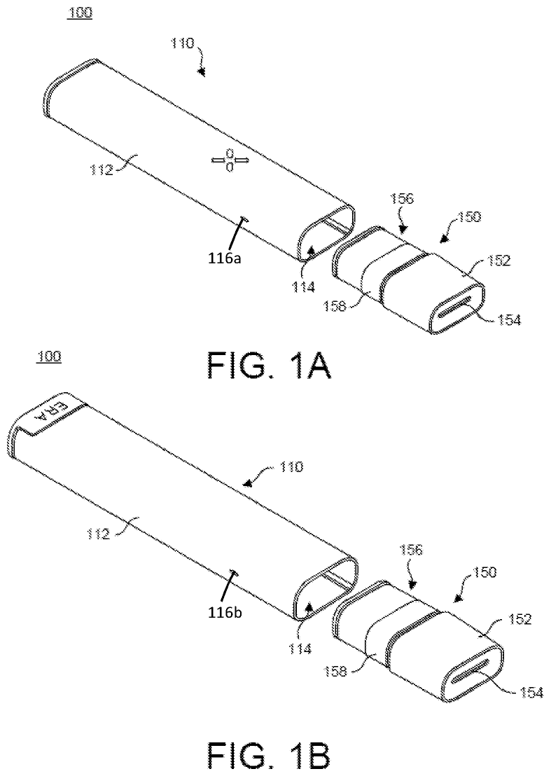

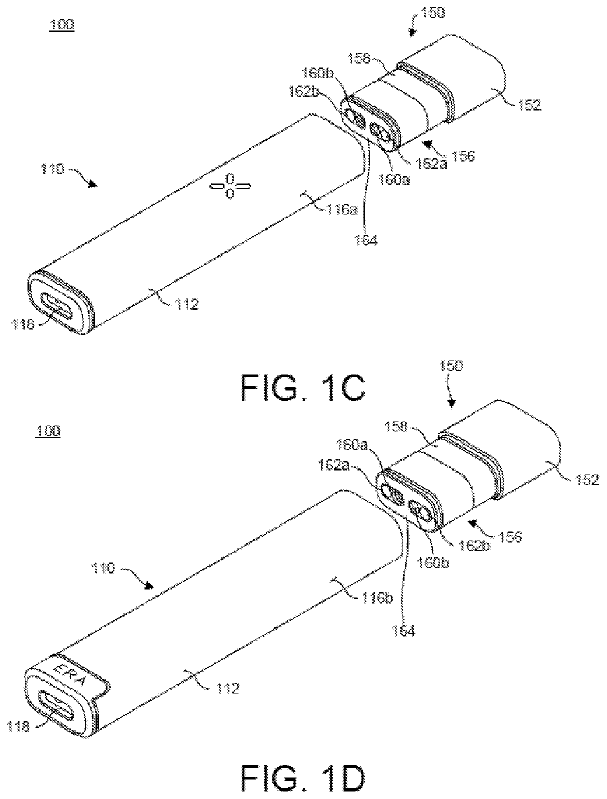

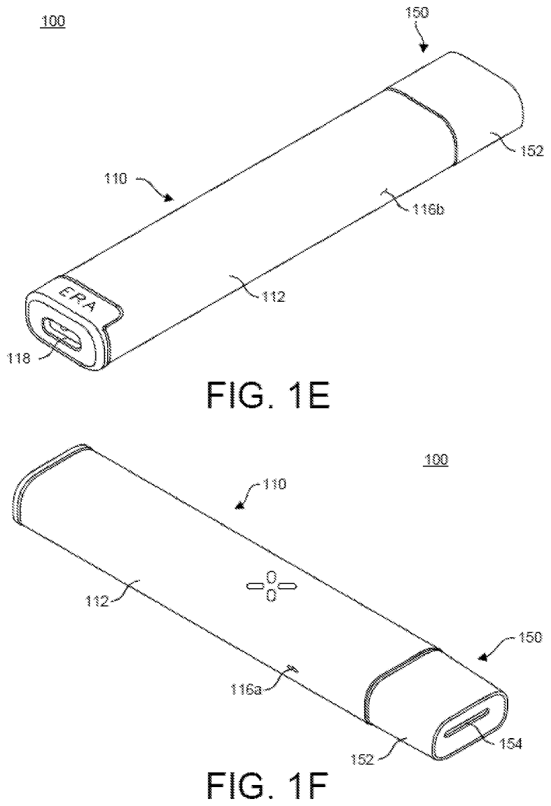

FIG. 1A-FIG. 1F illustrates features of a vaporizer device 100 including a vaporizer body 110 and a cartridge 150 consistent with implementations of the current subject matter. FIG. 1A is a bottom perspective view, and FIG. 1B is a top perspective view of the vaporizer device 100 with the cartridge 150 separated from a cartridge receptacle 114 on the vaporizer body 110. Both of the views in FIG. 1A and FIG. 1B are shown looking towards a mouthpiece 152 of the cartridge 150. FIG. 1C is a bottom perspective view, and FIG. 1D is a top perspective view of the vaporizer device with the cartridge 150 separated from the cartridge receptacle 114 of the vaporizer body 110. FIG. 1C and FIG. 1D are shown looking toward the distal end of the vaporizer body 110. FIG. 1E is top perspective view, and FIG. 1F is a bottom perspective view of the vaporizer device 100 with the cartridge 150 engaged for use with the vaporizer body 110.

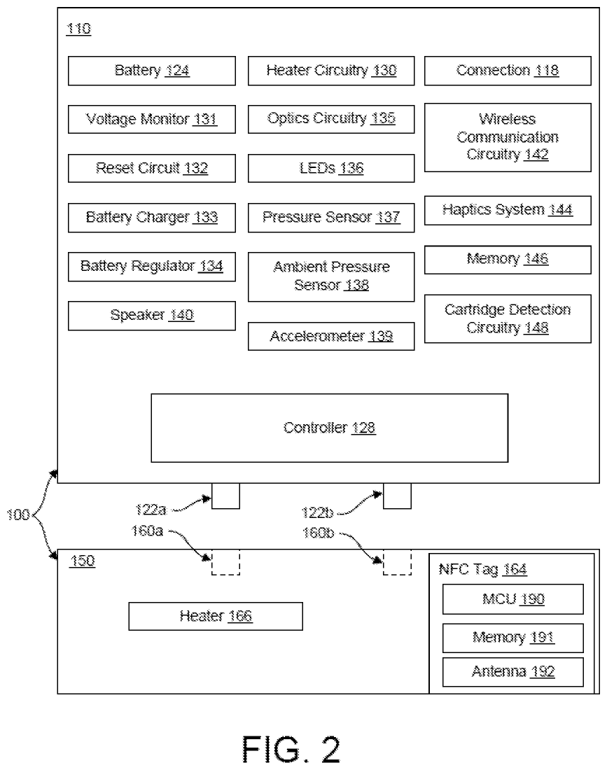

As shown in FIG. 1A-FIG. 1D, the cartridge 150 includes, at the proximal end, a mouthpiece 152 that is attached over a cartridge body 156 that forms a reservoir or tank 158 that holds a vaporizable material. The cartridge body 156 may be transparent, translucent, opaque, or a combination thereof. The mouthpiece 152 may include one or more openings 154 (see FIG. 1A, FIG. 1B, FIG. 1F) at the proximal end out of which vapor may be inhaled, by drawing breath through the vaporizer device 100. The distal end of the cartridge body 156 may couple to and be secured to the vaporizer body 110 within the cartridge receptacle 114 of the vaporizer body 110. Power pin receptacles 160a,b (see FIG. 1C, FIG. 1D) of the cartridge 150 mate with respective power pins or contacts 122a,b (see, for example, FIG. 2) of the vaporizer body 110 that extend into the cartridge receptacle 114. The cartridge 150 also includes air flow inlets 162a,b on the distal end of the cartridge body 156.

A tag 164, such as a data tag, a near-field communication (NFC) tag, or other type of wireless transceiver or communication tag, may be positioned on at least a portion of the distal end of the cartridge body 156. As shown in FIG. 1C and FIG. 1D, the tag 164 may substantially surround the power pin receptacles 160a,b and the air flow inlets 162a,b, although other configurations of the tag 164 may be implemented as well. For example, the tag 164 may be positioned between the power pin receptacle 160a and the power pin receptacle 160b, or the tag 164 may be shaped as a circle, partial circle, oval, partial oval, or any polygonal shape encircling or partially encircling the power pin receptacles 160a,b and the air flow inlets 162a,b or a portion thereof.

In the example of FIG. 1A, the vaporizer body 110 has an outer shell or cover 112 that may be made of various types of materials, including for example aluminum (e.g., AL6063), stainless steel, glass, ceramic, titanium, plastic (e.g., Acrylonitrile Butadiene Styrene (ABS), Nylon, Polycarbonate (PC), Polyethersulfone (PESU), and the like), fiberglass, carbon fiber, and any hard, durable material. The proximal end of the vaporizer body 110 includes an opening forming the cartridge receptacle 114, and the distal end of the vaporizer body 110 includes a connection 118, such as, for example, a universal serial bus Type C (USB-C) connection and/or the like. The cartridge receptacle 114 portion of the vaporizer body 110 includes one or more openings (air inlets) 116a,b that extend through the outer shell 112 to allow airflow therein, as described in more detail below. The vaporizer body 110 as shown has an elongated, flattened tubular shape that is curvature-continuous, although the vaporizer body 110 is not limited to such a shape. The vaporizer body 110 may take the form of other shapes, such as, for example, a rectangular box, a cylinder, and the like.

The cartridge 150 may fit within the cartridge receptacle 114 by a friction fit, snap fit, and/or other types of secure connection. The cartridge 150 may have a rim, ridge, protrusion, and/or the like for engaging a complimentary portion of the vaporizer body 110. While fitted within the cartridge receptacle 114, the cartridge 150 may be held securely within but still allow for being easily withdrawn to remove the cartridge 150.

Although FIG. 1A-FIG. IF illustrate a certain configuration of the vaporizer device 100, the vaporizer device 100 may take other configurations as well.

FIG. 2 is a schematic block diagram illustrating components of the vaporizer device 100 having the cartridge 150 and the vaporizer body 110 consistent with implementations of the current subject matter. Included in the vaporizer body 110 is a controller 128 that includes at least one processor and/or at least one memory configured to control and manage various operations among the components of the vaporizer device 100 described herein.

Heater control circuitry 130 of the vaporizer body 110 controls a heater 166 of the cartridge 150. The heater 166 may generate heat to provide vaporization of the vaporizable material. For example, the heater 166 may include a heating coil (e.g., a resistive heater) in thermal contact with a wick which absorbs the vaporizable material.

A battery 124 is included in the vaporizer body 110, and the controller 128 may control and/or communicate with a voltage monitor 131 which includes circuitry configured to monitor the battery voltage, a reset circuit 132 configured to reset (e.g., shut down the vaporizer device 100 and/or restart the vaporizer device 100 in a certain state), a battery charger 133, and a battery regulator 134 (which may regulate the battery output, regulate charging/discharging of the battery, and provide alerts to indicate when the battery charge is low, etc.).

The power pins 122a,b of the vaporizer body 110 engage the complementary power pin receptacles 160a,b of the cartridge 150 when the cartridge 150 is engaged with the vaporizer body 110. Alternatively, power pins may be part of the cartridge 150 for engaging complementary power pin receptacles of the vaporizer body 110. The engagement allows for the transfer of energy from an internal power source (e.g., the battery 124) to the heater 166 in the cartridge 150. The controller 128 may regulate the power flow (e.g., an amount or current and/or a voltage amount) to control a temperature at which the heater 166 heats the vaporizable material contained in the reservoir 158. According to implementations of the current subject matter, a variety of electrical connectors other than a pogo-pin and complementary pin receptacle configuration may be used to electrically connect the vaporizer body 110 and the cartridge 150, such as for example, a plug and socket connector.

The controller 128 may control and/or communicate with optics circuitry 135 (which controls and/or communicates with one or more displays such as LEDs 136 which may provide user interface output indications), a pressure sensor 137, an ambient pressure sensor 138, an accelerometer 139, and/or a speaker 140 configured to generate sound or other feedback to a user.

The pressure sensor 137 may be configured to sense a user drawing (i.e., inhaling) on the mouthpiece 152 and activate the heater control circuitry 130 of the vaporizer body 110 to accordingly control the heater 166 of the cartridge 150. In this way, the amount of current supplied to the heater 166 may be varied according the user's draw (e.g., additional current may be supplied during a draw, but reduced when there is not a draw taking place). The ambient pressure sensor 138 may be included for atmospheric reference to reduce sensitivity to ambient pressure changes and may be utilized to reduce false positives potentially detected by the pressure sensor 137 when measuring draws from the mouthpiece 152.

The accelerometer 139 (and/or other motion sensors, capacitive sensors, flow sensors, strain gauge(s), or the like) may be used to detect user handling and interaction, for example, to detect movement of the vaporizer body 110 (such as, for example, tapping, rolling, and/or any other deliberate movement associated with the vaporizer body 110).

The vaporizer body 110, as shown in FIG. 2, includes wireless communication circuity 142 that is connected to and/or controlled by the controller 128. The wireless communication circuity 142 may include a near-field communication (NFC) antenna that is configured to read from and/or write to the tag 164 of the cartridge 150. Alternatively or additionally, the wireless communication circuity 142 may be configured to automatically detect the cartridge 150 as it is being inserted into the vaporizer body 110. In some implementations, data exchanges between the vaporizer body 110 and the cartridge 150 take place over NFC. In some implementations, data exchanges between the vaporizer body 110 and the cartridge 150 may take place via a wired connection such as various wired data protocols.

The wireless communication circuitry 142 may include additional components including circuitry for other communication technology modes, such as Bluetooth circuitry, Bluetooth Low Energy circuitry, Wi-Fi circuitry, cellular (e.g., LTE, 4G, and/or 5G) circuitry, and associated circuitry (e.g., control circuitry), for communication with other devices. For example, the vaporizer body 110 may be configured to wirelessly communicate with a remote processor (e.g., a smartphone, a tablet, a computer, wearable electronics, a cloud server, and/or processor based devices) through the wireless communication circuitry 142, and the vaporizer body 110 may through this communication receive information including control information (e.g., for setting temperature, resetting a dose counter, etc.) from and/or transmit output information (e.g., dose information, operational information, error information, temperature setting information, charge/battery information, etc.) to one or more of the remote processors.

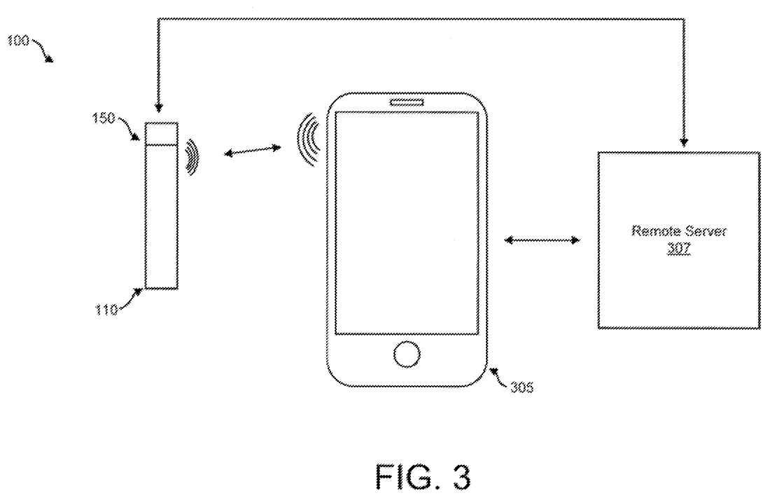

The tag 164 may be a type of wireless transceiver and may include a microcontroller unit (MCU) 190, a memory 191, and an antenna 192 (e.g., an NFC antenna) to perform the various functionalities described below with further reference to FIG. 3. NFC tag 164 may be, for example, a 1 Kbit or a 2Kbit tag that is of type ISO/IEC 15693. NFC tags with other specifications may also be used. The tag 164 may be implemented as active NFC, enabling reading and/or writing information via NFC with other NFC compatible devices including a remote processor, another vaporizer device, and/or wireless communication circuitry 142. Alternatively, the tag 164 may be implemented using passive NFC technology, in which case other NFC compatible devices (e.g., a remote processor, another vaporizer device, and/or wireless communication circuitry 142) may only be able to read information from the tag 164.

The vaporizer body 110 may include a haptics system 144, such as an actuator, a linear resonant actuator (LRA), an eccentric rotating mass (ERM) motor, or the like that provide haptic feedback such as a vibration as a “find my device” feature or as a control or other type of user feedback signal. For example, using an app running on a user device (such as, for example, a user device 305 shown in FIG. 3), a user may indicate that he/she cannot locate his/her vaporizer device 100. Through communication via the wireless communication circuitry 142, the controller 128 sends a signal to the haptics system 144, instructing the haptics system 144 to provide haptic feedback (e.g., a vibration). The controller 128 may additionally or alternatively provide a signal to the speaker 140 to emit a sound or series of sounds. The haptics system 144 and/or speaker 140 may also provide control and usage feedback to the user of the vaporizer device 100; for example, providing haptic and/or audio feedback when a particular amount of a vaporizable material has been used or when a period of time since last use has elapsed. Alternatively or additionally, haptic and/or audio feedback may be provided as a user cycles through various settings of the vaporizer device 100. Alternatively or additionally, the haptics system 144 and/or speaker 140 may signal when a certain amount of battery power is left (e.g., a low battery warning and recharge needed warning) and/or when a certain amount of vaporizable material remains (e.g., a low vaporizable material warning and/or time to replace the cartridge 150). Alternatively or additionally, the haptics system 144 and/or speaker 140 may also provide usage feedback and/or control of the configuration of the vaporizer device 100 (e.g., allowing the change of a configuration, such as target heating rate, heating rate, etc.).

The vaporizer body 110 may include circuitry for sensing/detecting when a cartridge 150 is connected and/or removed from the vaporizer body 110. For example, cartridge-detection circuitry 148 may determine when the cartridge 150 is connected to the vaporizer body 110 based on an electrical state of the power pins 122a,b within the cartridge receptacle 114. For example, when the cartridge 150 is present, there may be a certain voltage, current, and/or resistance associated with the power pins 122a,b, when compared to when the cartridge 150 is not present. Alternatively or additionally, the tag 164 may also be used to detect when the cartridge 150 is connected to the vaporizer body 110.

The vaporizer body 110 also includes the connection (e.g., USB-C connection, micro-USB connection, and/or other types of connectors) 118 for coupling the vaporizer body 110 to a charger to enable charging the internal battery 124. Alternatively or additionally, electrical inductive charging (also referred to as wireless charging) may be used, in which case the vaporizer body 110 would include inductive charging circuitry to enable charging. The connection 118 at FIG. 2 may also be used for a data connection between a computing device and the controller 128, which may facilitate development activities such as, for example, programming and debugging, for example.

The vaporizer body 110 may also include a memory 146 that is part of the controller 128 or is in communication with the controller 128. The memory 146 may include volatile and/or non-volatile memory or provide data storage. In some implementations, the memory 146 may include 8 Mbit of flash memory, although the memory is not limited to this and other types of memory may be implemented as well.

FIG. 3 illustrates communication between the vaporizer device 100 (including the vaporizer body 110 and the cartridge 150), the user device 305 (e.g., a smartphone, tablet, laptop, and/or the like), and a remote server 307 (e.g., a server coupled to a network, a cloud server coupled to the Internet, and/or the like) consistent with implementations of the current subject matter. The user device 305 wirelessly communicates with the vaporizer device 100. A remote server 307 may communicate directly with the vaporizer device 100 or through the user device 305. The vaporizer body 110 may communicate with the user device 305 and/or the remote server 307 through the wireless communication circuitry 142. In some implementations, the cartridge 150 may establish through the tag 164 communication with the vaporizer body 110, the user device 305, and/or the remote server 307.

An application software (“app”) running on at least one of the remote processors (the user device 305 and/or the remote server 307) may be configured to control operational aspects of the vaporizer device 100 and receive information relating to operation of the vaporizer device 100. For example, the app may provide a user with capabilities to input or set desired properties or effects, such as, for example, a particular temperature or desired dose, which is then communicated to the controller 128 of the vaporizer body 110 through the wireless communication circuitry 142. The app may also provide a user with functionality to select one or more sets of suggested properties or effects that may be based on the particular type of vaporizable material in the cartridge 150. For example, the app may allow adjusting heating based on the type of vaporizable material, the user's (of the vaporizer device 100) preferences or desired experience, and/or the like.

Data read from the tag 164 from the wireless communication circuitry 142 of the vaporizer body 110 may be transferred to one or more of the remote processors (e.g., the user device 305 and/or the remote server 307) to which it is connected, which allows for the app running on the one or more processors to access and utilize the read data for a variety of purposes. For example, the read data relating to the cartridge 150 may be used for providing recommended temperatures, session control, usage tracking, and/or assembly information.

The cartridge 150 may also communicate directly, through the tag 164, with other devices. This enables data relating to the cartridge 150 to be written to/read from the tag 164, without interfacing with the vaporizer body 110. The tag 164 thus allows for identifying information (e.g., pod ID, batch ID, etc.) related to the cartridge 150 to be associated with the cartridge 150 by one or more remote processors. For example, when the cartridge 150 is filled with a certain type of vaporizable material, this information may be transmitted to the tag 164 by filling equipment. Then, the vaporizer body 110 is able to obtain this information from the tag 164 (e.g., via the wireless communication circuity 142 at the vaporizer body 110) to identify the vaporizable material currently being used and accordingly adjust the controller 128 based on, for example, user-defined criteria or pre-set parameters associated with the particular type of vaporizable material (set by a manufacturer or as determined based upon user experiences/feedback aggregated from other users). For example, a user may establish (via the app) a set of criteria relating to desired effects for or usage of one or more types of vaporizable materials. When a certain vaporizable material is identified, based on communication via the tag 164, the controller 128 may accordingly adopt the established set of criteria, which may include, for example, temperature and dose, for that particular vaporizable material.

Consistent with implementations of the current subject matter, the vaporizable material used with the vaporizer device may be provided within the cartridge. The vaporizer device may be a cartridge-using vaporizer device, a cartridge-less vaporizer device, or a multi-use vaporizer device capable of use with or without a cartridge. For example, a multi-use vaporizer device may include a heating chamber (e.g., an oven) configured to receive the vaporizable material directly in the heating chamber and also configured to receive the cartridge having a reservoir or the like for holding the vaporizable material. In various implementations, the vaporizer device may be configured for use with liquid vaporizable material (e.g., a carrier solution in which an active and/or inactive ingredient(s) are suspended or held in solution or a liquid form of the vaporizable material itself) or solid vaporizable material. Solid vaporizable material may include a plant material that emits some part of the plant material as the vaporizable material (e.g., such that some part of the plant material remains as waste after the vaporizable material is emitted for inhalation by a user) or optionally may be a solid form of the vaporizable material itself such that all of the solid material may eventually be vaporized for inhalation. Liquid vaporizable material may likewise be capable of being completely vaporized or may include some part of the liquid material that remains after all of the material suitable for inhalation has been consumed.

Aspects of the current subject matter relate to controlling the temperature of the vaporizer device 100 including determining, based at least on an ambient pressure, a setpoint temperature of the vaporizer device 100 and/or a ramp rate for achieving the setpoint temperature of the vaporizer device 100. As used herein, “setpoint temperature” may refer to the temperature at which the vaporizer device 100, for example, the heater 166, operates to vaporize the vaporizable material contained in the cartridge 150. When vaporized, the vaporizable material in the cartridge 150 may undergo a phase transition, for example, from a liquid to a vapor. Meanwhile, “ramp rate” may refer to the rate at which the temperature of the vaporizer device 100, for example, the heater 166, is changed in order to achieve the setpoint temperature.

A user may have preferences for the flavor profile of the vapor experienced by the user. However, the setpoint temperature of the vaporizer device 100 may affect the strength of vapor and thus the flavor profile of the vapor experienced by a user. For example, subjecting the vaporizable material to a higher setpoint temperature may produce a denser vapor with a greater mass of aerosol and/or total particulate matter. By contrast, subjecting the vaporizable material to a lower setpoint temperature may produce a less dense vapor with a lesser mass of aerosol and/or total particulate matter. It should be appreciated that a denser vapor may be associated with a more concentrated flavor while the less dense vapor may be associated with more nuanced flavors.

Fluctuations in the ambient pressure around the vaporizer device 100 may determine the boiling point of the vaporizable material contained in the cartridge 150. The boiling point of the vaporizable material may correspond to the temperature at which the vaporizable material undergoes a phase transition, for example, from a liquid to a vapor. To further illustrate, Equation (1) below characterizes the phase transitions of the vaporizable material:

$$\frac{Δ P}{Δ T} = \frac{L}{T Δ v} = \frac{Δ s}{Δ v} \qquad ( 1 )$$

wherein

$$\frac{Δ P}{Δ T}$$

may correspond to the slope of a coexistence curve separating the two different phases of the vaporizable material, L may denote the specific latent heat of the vaporizable material, Δs may denote the specific entropy change of the phase transition of the vaporizable material, and Δv may denote the specific volume change of the phase transition of the vaporizable material.

Based on Equation (1), the temperature T at which the vaporizable material undergoes a phase transition (e.g., from liquid to solid) when subject to an ambient pressure P may be determined by applying Equation (2) below:

$$ln ( \frac{{P}_{2}}{{P}_{1}} ) = \frac{L}{R} ( \frac{1}{{T}_{1}} - \frac{1}{{T}_{2}} ) \qquad ( 2 )$$

wherein R may denote the specific gas constant associated with the vaporizable material, P₁ may denote a first pressure, P₂ may denote a second pressure, T₁ may denote a first temperature at which the vaporizable material undergoes a phase change at the first pressure P₁, and T₂ may denote a second temperature at which the vaporizable material undergoes a phase change at the second pressure P₂. It should be appreciated that the specific latent heat L and the specific gas constant R of the vaporizable material may be dependent on the type of the vaporizable material contained in the cartridge 150.

Fluctuations in ambient pressure and humidity may also affect the flavor profile of the vapor experienced by the user due to changes in the sensitivity of the human taste bud in different ambient pressure and/or humidity environments. If the vaporizer device 100 operates at the same setpoint temperature in different ambient pressure and/or humidity environments, the vapor experienced by the user may be inconsistent with the preferred flavor profile of the user. As such, in some example embodiments, the controller 128 may adjust, based at least on the ambient pressure, the setpoint temperature of the vaporizer device 100 (e.g., the heater 166) in order to maintain a consistently optimal user experience. For example, because the sensitivity of human taste buds is diminished in a low ambient pressure and/or low humidity environment, the user in a low ambient pressure and/or low humidity environment may experience a vapor that is less potent than the user's preferred flavor profile. Accordingly, when the user is in a low ambient pressure and/or low humidity environment, the setpoint temperature of the vaporizer device 100 may be adjusted to produce a denser vapor with a more concentrated flavor that compensates for the diminished sensitivity of the user's taste buds. Alternatively, to prevent the user from experiencing a vapor that is too potent for the user's preferences, the setpoint temperature of the vaporizer device 100 may be adjusted to produce a less dense vapor with a more nuanced flavor when the user is in a higher ambient pressure and/or humidity environment in which the user's taste buds are more sensitive.

In some example embodiments, the setpoint temperature of the vaporizer device 100 may be adjusted based at least on the ambient pressure around the vaporizer device 100 measured, for example, by the ambient pressure sensor 138. By adjusting the setpoint temperature of the vaporizer device 100 based on ambient pressure, the vapor experienced by the user in varying ambient pressure and/or humidity environments may remain consistent with the user's preferred flavor profile. For example, when measurements from the ambient pressure senor 138 indicate a decrease in ambient pressure, the setpoint temperature of the vaporizer device 100 may be increased in order for the vaporizer device 100 to produce a denser vapor with a more concentrated flavor. Alternatively, when measurements from the ambient pressure senor 138 indicate an increase in ambient pressure, the setpoint temperature of the vaporizer device 100 may be decreased in order for the vaporizer device 100 to produce a less dense vapor with a less concentrated flavor.

Referring again to Equations (1) and (2), the setpoint temperature of the vaporizer device 100 at an ambient pressure P may correspond to the temperature T at which a vaporizable material undergoes a phase transition (e.g., from liquid to vapor) at the ambient pressure P. The controller 128 may adjust the setpoint temperature of the vaporizer device 100 to be equal to the changing temperatures T at which the vaporizable material undergoes the phase transition at different ambient pressures P. As one example, the controller 128 may adjust the setpoint temperature of the vaporizer device 100 to 100° C. when the vaporizer device 100 is operating at sea level and the ambient pressure is approximately 14.7 pounds per square inch (PSI). When the vaporizer device 100 is operating at 1.6 kilometers above sea level and is exposed to an atmospheric pressure of approximately 12 pounds per square inch (PSI), the controller 128 may adjust the setpoint temperature of the vaporizer device 100 to 95° C.

Furthermore, the controller 128 may adjust the setpoint temperature of the vaporizer device 100 at the ambient pressure P relative to the temperature T at which the vaporizable material undergoes the phase transition at the ambient pressure P in order for the vaporizer device 100 to produce a vapor that is consistent with the preferred flavor profile of the user. For instance, to compensate for the diminished sensitivity of the user's taste buds in a lower ambient pressure and/or humidity environment, the setpoint temperature of the vaporizer device 100 may be adjusted to exceed the temperature T at which the vaporizable material undergoes the phase transition at the ambient pressure P in order for the vaporizer device 100 to produce a denser vapor with a more concentrated flavor.

In some example embodiments, the setpoint temperature of the vaporizer device 100 may be adjusted, based at least on the ambient pressure around the vaporizer device 100, relative to a preset setpoint temperature. The preset setpoint temperature may be determined based on a variety of factors including, for example, user preferences, crowdsourced information, type of vaporizable material (e.g., composition of the vaporizable material and/or the like), and/or the like. Nevertheless, the preset setpoint temperature may be determined for a first ambient pressure whereas the vaporizer device 100 is subject to a second ambient pressure. The preset setpoint temperature may be a default setting (e.g., a default provided by the vaporizer device 100, a default corresponding to the vaporizable material (e.g., a strain of cannabinoids), or a default corresponding to the cartridge 150). The preset setpoint temperature may carry over from a previous use of the vaporizer device 100 and/or the cartridge 150. The preset setpoint temperature may be defined and/or updated by the user using the app running on the user device 305.

In some example embodiments, the setpoint temperature of the vaporizer device 100 may be adjusted relative to the temperature at which one or more vaporizable materials undergo a phase transition (e.g., from liquid to vapor) at the ambient pressure P. For example, the setpoint temperature of the vaporizer device 100 may be adjusted to achieve targeted vaporization of a first vaporizable material (e.g., cannabidiol (CBD)) that undergoes a phase transition at the first temperature T₁. The first temperature T₁ may be lower than the second temperature T₂ at which a second vaporizable material (e.g., tetrahydrocannabinol (THC)) undergoes a phase transition. Accordingly, to achieve targeted vaporization of the first vaporizable material, the setpoint temperature of the vaporizer device 100 may be adjusted to be higher than the first temperature T₁ but lower than the second temperature T₂.

For example, the preset setpoint temperature may be the most recent setpoint temperature used with the cartridge 150 and/or the vaporizer body 110. For example, the most recent setpoint temperature may be stored in the memory 146 of the vaporizer body 110 and associated with the cartridge 150 based on an identifier of the cartridge 150 that is read by the controller 128 from the tag 164 of the cartridge 150. When the controller 128, through the wireless communication circuitry 142, recognizes the cartridge 150, the controller 128 may access from the memory 146 the most recent setpoint temperature and use this as the preset setpoint temperature.

As an additional example, the preset setpoint temperature may be associated with the cartridge 150 based on various factors, such as the type of vaporizable material (e.g., a manufacturer defines the preset setpoint temperature for the cartridge 150). The preset setpoint temperature may be stored on the tag 164 of the cartridge 150 and read by the controller 128.

According to some example implementations, the preset setpoint temperature of the vaporizer device 100 may be established in response to one or more user actions including, for example, the cartridge 150 being inserted into and/or removed from the vaporizer body 110. In particular, when the cartridge 150 is inserted into the vaporizer body 110, the cartridge 150 is detected by, for example, the cartridge detection circuitry 148 or by other detection means. Upon detection of the cartridge 150, the vaporizer body 110 may determine or identify the preset setpoint temperature as described above. For example, the controller 128 of the vaporizer body 110 may use a default preset setpoint temperature, the preset setpoint temperature associated with the cartridge 150 and/or the vaporizer body 110, the most recent setpoint temperature used with the cartridge 150 and/or the vaporizer body 110, and/or a user defined preset setpoint temperature. In some implementations of the current subject matter, one or more of the preset setpoint temperature options may take priority over the others. For example, the default preset setpoint temperature may have a low priority such that the preset setpoint temperature associated with the cartridge 150 takes precedence in setting the preset setpoint temperature. The user defined preset setpoint temperature may override the other preset setpoint temperature options. The priority may be predefined, user-defined, and/or user- adjustable.

In some example implementations, the ramp rate of the vaporizer device 100 may be adjusted based at least on the ambient pressure around the vaporizer device 100 measured, for example, by the ambient pressure sensor 138. As noted, fluctuations in the ambient pressure may determine the boiling point of the vaporizable material contained in the cartridge 150. That is, the vaporizable material in the cartridge 150 may undergo a phase transition (e.g., from liquid to vapor) at different temperatures depending on the ambient pressure around the vaporizer device 100. Accordingly, the ramp rate of the vaporizer device 100 may be adjusted in order to compensate for the changes in the boiling point of the vaporizable material contained in the cartridge 150. For instance, increasing the ramp rate of the vaporizer device 100 may decrease the amount of time necessary for the vaporizer device 100 to reach the setpoint temperature of the vaporizer device 100. The adjusting of the ramp rate may therefore prevent the user from experiencing a time lag when using the vaporizer device 100 in a higher ambient pressure environment in which the vaporizable material in the cartridge 150 undergoes the phase transition at a higher temperature. For example, the ramp rate of the vaporizer device 100 may be adjusted such that the vaporizer device 100 achieves the higher boiling point of the vaporizable material in a higher ambient pressure environment in a substantially same amount of time as the vaporizer device 100 achieves a lower boiling point of the vaporizable material in a lower ambient pressure environment. That is, the ramp rate of the vaporizer device 100 may be adjusted such that the vaporizer device 100 achieves the higher boiling point of the vaporizer material in a same or a similar amount of time as the vaporizer device 100 achieves the lower boiling point of the vaporizable material.

Referring again to FIG. 2, the controller 128 may apply a proportional-integral-derivative (PID) control technique when adjusting the temperature of the heater 166 to achieve the setpoint temperature of the vaporizer device 100. As such, the controller 128 may continuously calculate an error corresponding to a difference between the setpoint temperature of the vaporizer device 100 and the current temperature of the vaporizer device 100, and apply a correction based on a proportional term, an integral term, and a derivative term. In some example implementations, the ramp rate of the vaporizer device 100 may be adjusted by adjusting at least one of the proportional term, the integral term, or the derivative term of the proportional-integral-derivative control, which generates an output value that is proportional to the current error value determined by the controller 128.

For example, the controller 128 may adjust the temperature of the heater 166, including by starting or stopping the discharge of the battery 124 to the heater 166, based on an error in the current temperature of the heater 166 relative to the setpoint temperature. It should be appreciated that the temperature of the heater 166 may correspond to a resistance through the heater 166 (e.g., through a heating coil). That is, the temperature of the heater 166 may be correlated to the resistance through the heater 166 by a thermal coefficient of resistance associated with the heater 166. As such, the current resistance through the heater 166 may correspond to the current temperature of the heater 166 while the target resistance through the heater 166 may correspond to the setpoint temperature of the heater 166. Moreover, the controller 128 may start or stop the discharge of the battery 124 to the heater 166 based on an error in the current resistance through the heater 166 relative to a target resistance.

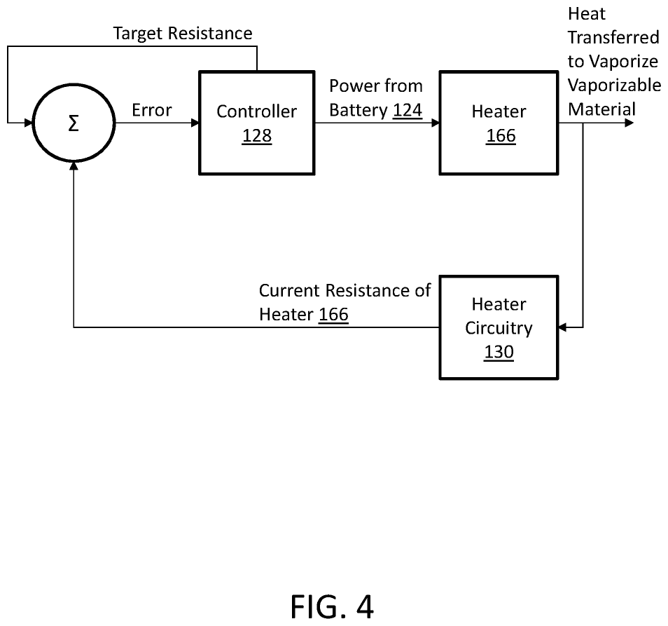

To further illustrate, FIG. 4 depicts a block diagram illustrating an example of proportional-integral-derivative (PID) control, in accordance with some implementations of the current subject matter. As shown in FIG. 4, the controller 128 may control the discharge of the battery 124 to the heater 166 of the cartridge 150. Meanwhile, the flow of current from the battery 124 through the heater 166 may generate heat, for example, through resistive heating. The heater 166 may include a heating coil (e.g., a resistive heater) in thermal contact with a wick which absorbs the vaporizable material. The heat generated by the heating coil may be transferred to the wick, which may be in thermal contact with the heating coil. For instance, the heat that is generated by the heating coil may be transferred to the wick through conductive heat transfer, convective heat transfer, radiative heat transfer, and/or the like. The heat from the heating coil may vaporize at least some of the vaporizable material held by the wick.

Referring again to FIG. 4, the heater circuitry 130 may be configured to determine a current resistance of the heater 166. As noted, the current resistance of the heater 166 may correspond to a current temperature of the heater 166. Accordingly, the controller 128, when applying a proportional-integral-derivative control technique, may adjust and/or maintain the temperature of the heater 166 based at least on an error between the current resistance of the heater 166 and a target resistance corresponding to the setpoint temperature for the heater 166. As shown in FIG. 4, the controller 128 may adjust, based at least on the error between the current resistance through the heater 166 and the target resistance, the discharge of the battery 124 to the heater 166. For example, the controller 128 may start the discharge of the battery 124 to the heater 166 if the current resistance of the heater 166 is below the target resistance. Alternatively or additionally, the controller 128 may stop the discharge of the battery 124 to the heater 166 if the current resistance of the heater 166 is equal to and/or above the target resistance.

The controller 128 may adjust, based at least on the error between the current resistance through the heater 166 and the target resistance, the discharge of the battery 124 to the heater 166 in order to achieve the target resistance corresponding to the setpoint temperature of the vaporizer device 100. It should be appreciated that the ramp rate at which the temperature of the heater 166 is changed in order to achieve the setpoint temperature of the vaporizer device 100 may be adjusted by at least adjusting the rate at which the battery 124 is discharged to the heating coil. Moreover, the controller 128 may adjust the discharge of the battery 124 based at least on a proportional term of the proportional-integral-derivative (PID) control being applied by the controller 128. As noted, the proportional term may generate an output value that is proportional to the current error value. For instance, a larger proportional term may increase the step size of the change towards achieving the setpoint temperature while a smaller proportional term may decrease the step size of the change towards achieving the setpoint temperature. Accordingly, the controller 128 may adjust the proportional term in order to adjust, based at least on the ambient pressure around the vaporizer device 100, the ramp rate at which the temperature of the heater 166 is changed in order to achieve the setpoint temperature of the vaporizer device 100. However, it should be appreciated that the controller 128 may also adjust the integral term and/or the derivative term in order to adjust the ramp rate of the vaporizer device 100.

In some example implementations, the controller 128 may be configured to adjust the setpoint temperature of the vaporizer device 100 and/or the ramp rate for achieving the setpoint temperature in response to detecting a change in ambient pressure. For example, the controller 128 may detect the change in ambient pressure based at least on one or more measurements from the ambient pressure sensor 138. Alternatively and/or additionally, the controller 128 may detect the change in ambient pressure based on a change in the location of the vaporizer device 100. The change in the location of the vaporizer device 100 may be associated with a change in the altitude of the vaporizer device 100 as well as a corresponding change in the ambient pressure around the vaporizer device 100.

In some example embodiments, the controller 128 may be configured to adjust the setpoint temperature of the vaporizer device 100 and/or the ramp rate for achieving the setpoint temperature automatically, with or without notifying the user of the adjustments. Alternatively, the controller 128 may respond to detecting the change in the ambient pressure by sending, to the user, a notification that the setpoint temperature and/or the ramp rate of the vaporizer device 100 requires adjustment. For example, the notification may be sent to the user device 305 and displayed by the application software running on the user device 305. Moreover, the notification may include a recommendation for the user to adjust the setpoint temperature and/or the ramp rate of the vaporizer device 100. The user may respond to the notification by at least making the recommended adjustments to the setpoint temperature and/or ramp rate of the vaporizer device 100. Alternatively and/or additionally, the notification may prompt the user to consent to the adjustments to the setpoint temperature and/or the ramp rate of the vaporizer device 100, as determined by the controller 128.

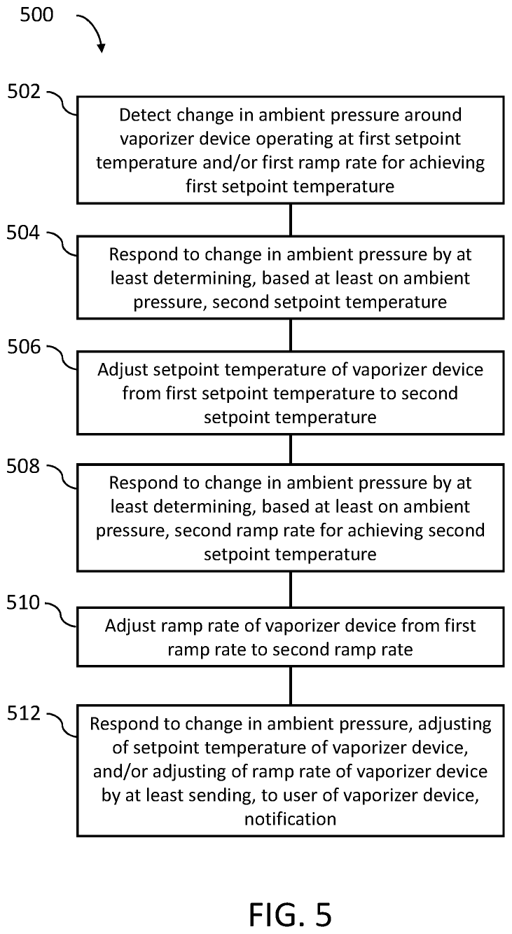

FIG. 5 depicts a chart illustrating a process 500 for adjusting the setpoint temperature and/or ramp rate of a vaporizer device, in accordance with some example embodiments. Referring to FIG. 1A-FIG. 5, the process 500 may be performed by the vaporizer device 100, for example, by the controller 128.

At 502, the controller 128 may detect a change in an ambient pressure around the vaporizer device 100 operating at a first setpoint temperature and/or a first ramp rate for achieving the first setpoint temperature. For example, the controller 128 may detect the change in ambient pressure based at least on one or more measurements from the ambient pressure sensor 138. Alternatively and/or additionally, the controller 128 may detect the change in ambient pressure based on a change in the location of the vaporizer device 100, which may indicate a change in the altitude of the vaporizer device 100 as well as a corresponding change in the ambient pressure around the vaporizer device 100.

At 504, the controller 128 may respond to the change in the ambient pressure by at least determining, based at least on the ambient pressure, a second setpoint temperature. At 506, the controller 128 may adjust a setpoint temperature of the vaporizer device 100 from the first setpoint temperature to the second setpoint temperature. The boiling point of the vaporizable material contained in the cartridge 150 (e.g., the temperature at which the vaporizable material undergoes a phase transition from liquid to vapor) may change due to fluctuations in the ambient pressure around the vaporizer device 100. As such, in some example embodiments, in response to detecting a change from a first ambient pressure P₁ associated with a first boiling point T₁ to a second ambient pressure P₂ associated with a second boiling point T₂, the controller 128 may adjust the setpoint temperature of the vaporizer device 100 to at least correspond to the second boiling point T₂ associated with the second ambient pressure P₂.

As noted, fluctuations in ambient pressure and humidity may also affect the flavor profile of the vapor experienced by the user of the vaporizer device 100 due to changes in the sensitivity of the human taste bud in different ambient pressure and/or humidity environments. Accordingly, in some example embodiments, the controller 128 may further adjust the setpoint temperature of the vaporizer device 100 relative to the second boiling point T₂ in order to ensure that the vapor experienced by the user as the ambient pressure around the vaporizer device 100 changes from the first ambient pressure P₁ to the second ambient pressure P₂ remains consistent with the user's preferred flavor profile.

In some example embodiments, the controller 128 may automatically adjust the setpoint temperature of the vaporizer device 100 (e.g., from the first setpoint temperature T₁ to the second setpoint temperature T₂) with or without notifying the user of the change. That is, the controller 128 may adjust the ambient pressure of the vaporizer device 100 without any input from the user of the vaporizer device 100. Alternatively, the controller 128 may adjust the setpoint temperature of the vaporizer device 100 in response to one or more inputs from the user of the vaporizer device 100. The one or more inputs from the user may include an indication adjusting, to the second setpoint temperature T₂, the setpoint temperature of the vaporizer device. Alternatively and/or additionally, the one or more inputs from the user may include an indication consenting to adjust the setpoint temperature of the vaporizer device 100 to the second setpoint temperature T₂.

At 508, the controller 128 may respond to the change in the ambient pressure by at least determining, based at least on the ambient pressure, a second ramp rate for achieving the second setpoint temperature. In some example embodiments, the ramp rate of the vaporizer device 100 may be adjusted based on the ambient pressure around the vaporizer device 100 in order to compensate for the changes in the boiling point of the vaporizable material contained in the cartridge 150 due to fluctuations in the ambient pressure around the vaporizer device 100. For example, if the vaporizer device 100 operates at a first ramp rate to achieve the first setpoint temperature T₁ at the first ambient pressure P₁, the vaporizer device 100 may be adjusted to operate at a second ramp rate to achieve the second setpoint temperature T₂ at the second ambient pressure P₂. Adjusting the ramp rate may, as noted, prevent the user from experiencing a time lag when using the vaporizer device 100 in a higher ambient pressure environment in which the vaporizable material in the cartridge 150 undergoes the phase transition at a higher temperature.

At 510, the controller 128 may adjust a ramp rate of the vaporizer device 100 from the first ramp rate to the second ramp rate. The controller 128 may, as noted, apply a proportional-integral-derivative (PID) control technique when adjusting the temperature of the heater 166 to achieve the setpoint temperature of the vaporizer device 100. For instance, the controller 128 may continuously calculate an error corresponding to a difference between the setpoint temperature of the vaporizer device 100 and the current temperature of the vaporizer device 100, and apply a correction based on a proportional term, an integral term, and a derivative term. Accordingly, in some implementations of the current subject matter, the controller 128 may adjust the ramp rate of the vaporizer device 100 by at least adjusting the proportional term, which generates an output value that is proportional to the current error value determined by the controller 128.

In some example embodiments, the controller 128 may automatically adjust the ramp rate of the vaporizer device 100 with or without notifying the user of the change. Alternatively, the controller 128 may adjust the ramp rate of the vaporizer device 100 in response to one or more inputs from the user of the vaporizer device 100. The one or more inputs from the user may include an indication to adjust, to the second ramp rate, the ramp rate of the vaporizer device. Alternatively and/or additionally, the one or more inputs from the user may include an indication consenting to adjust the ramp rate of the vaporizer device 100 to the second ramp rate.

At 512, the controller 128 may respond to the change in the ambient pressure, the adjusting of the setpoint temperature of the vaporizer device 100, and/or the adjusting of the ramp rate of the vaporizer device 100 by at least sending, to the user of the vaporizer device 100, a notification. The controller 128 may, as noted, adjust the setpoint temperature of the vaporizer device 100 and/or the ramp rate for achieving the setpoint temperature automatically, with or without notifying the user of the adjustments. Alternatively, the controller 128 may respond to detecting the change in the ambient pressure by sending, to the user, a notification that the setpoint temperature and/or the ramp rate of the vaporizer device 100 requires adjustment. For example, the notification may be sent to the user device 305 and displayed by the application software running on the user device 305. Moreover, the notification may include a recommendation for the user to adjust the setpoint temperature and/or the ramp rate of the vaporizer device 100. The user may respond to the notification by at least making the recommended adjustments to the setpoint temperature and/or ramp rate of the vaporizer device 100. Alternatively and/or additionally, the notification may prompt the user to consent to the adjustments to the setpoint temperature and/or the ramp rate of the vaporizer device 100, as determined by the controller 128.

In some examples, the vaporizable material may include a viscous liquid such as, for example a cannabis oil. In some variations, the cannabis oil comprises between 0.3% and 100% cannabis oil extract. The viscous oil may include a carrier for improving vapor formation, such as, for example, propylene glycol, glycerol, medium chain triglycerides (MCT) including lauric acid, capric acid, caprylic acid, caproic acid, etc., at between 0.01% and 25% (e.g., between 0. 1% and 22%, between 1% and 20%, between 1% and 15%, and/or the like). In some variations the vapor-forming carrier is 1,3-Propanediol. A cannabis oil may include a cannabinoid or cannabinoids (natural and/or synthetic), and/or a terpene or terpenes derived from organic materials such as for example fruits and flowers. For example, any of the vaporizable materials described herein may include one or more (e.g., a mixture of) cannabinoid including one or more of: CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), CBGM (Cannabigerol Monomethyl Ether), Tetrahydrocannabinol, Cannabidiol (CBD), Cannabinol (CBN), Tetrahydrocannabinolic Acid (THCA), Cannabidioloc Acid (CBDA), Tetrahydrocannabivarinic Acid (THCVA), one or more Endocannabinoids (e.g., anandamide, 2-Arachidonoylglycerol, 2-Arachidonyl glyceryl ether, N-Arachidonoyl dopamine, Virodhamine, Lysophosphatidylinositol), and/or a synthetic cannabinoids such as, for example, one or more of: JWH-018, JWH-073, CP-55940, Dimethylheptylpyran, HU-210, HU-331, SR144528, WIN 55,212-2, JWH-133, Levonantradol (Nantrodolum), and AM-2201. The oil vaporization material may include one or more terpene, such as, for example, Hemiterpenes , Monoterpenes (e.g., geraniol, terpineol, limonene, myrcene, linalool, pinene, Iridoids), Sesquiterpenes (e.g., humulene, farnesenes, farnesol), Diterpenes (e.g., cafestol, kahweol, cembrene and taxadiene), Sesterterpenes, (e.g., geranylfarnesol), Triterpenes (e.g., squalene), Sesquarterpenes (e.g, ferrugicadiol and tetraprenylcurcumene), Tetraterpenes (lycopene, gamma-carotene, alpha- and beta-carotenes), Polyterpenes, and Norisoprenoids. For example, an oil vaporization material as described herein may include between 0.3-100% cannabinoids (e.g., 0.5-98%, 10-95%, 20-92%, 30-90%, 40-80%, 50-75%, 60-80%, etc.), 0-40% terpenes (e.g., 1-30%, 10-30%, 10-20%, etc.), and 0-25% carrier (e.g., medium chain triglycerides (MCT)).

In any of the oil vaporizable materials described herein (including in particular, the cannabinoid-based vaporizable materials), the viscosity may be within a predetermined range. At room temperature of about 23° C., the range may be between about 30 cP (centipoise) and about 200 kcP (kilocentipoise). Alternatively, the range may be between about 30 cP and about 115 kcP. Alternatively, the range may be between about 40 cP and about 113 kcP. Alternatively, the range may be between about 50 cP and about 100 kcP. Alternatively, the range may be between about 75 cP and about 75 kcP. Alternatively, the range may be between about 100 cP and about 50 kcP. Alternatively, the range may be between about 125 cP and about 25 kcP. Outside of these ranges, the vaporizable material may fail in some instances to wick appropriately to form a vapor as described herein. In particular, it is typically desired that the oil may be made sufficiently thin to both permit wicking at a rate that is useful with the apparatuses described herein, while also limiting leaking. For example, viscosities below that of about 30 cP at room temperature might result in problems with leaking, and in some instances viscosities below that of about 100 cP at room temperature might result in problems with leaking.

Although the disclosure, including the figures, described herein may described and/or exemplify these different variations separately, it should be understood that all or some, or components of them, may be combined.

Although various illustrative embodiments are described above, any of a number of changes may be made to various embodiments. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as, for example, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, are possible.

In the descriptions above and in the claims, phrases such as, for example, “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A vaporizer device, comprising:
a pressure sensor configured to measure an ambient pressure; and
a controller configured to:
detect a change in the ambient pressure from a first ambient pressure to a second ambient pressure, the first ambient pressure associated with a first temperature at which a vaporizable material undergoes a phase transition, and the second ambient pressure associated with a second temperature at which the vaporizable material undergoes the phase transition; and
in response to detecting the change in the ambient pressure, adjust a setpoint temperature of the vaporizer device, the setpoint temperature being adjusted based at least on the second temperature at which the vaporizable material undergoes the phase transition at the second ambient pressure, and the vaporizer device operating at the setpoint temperature in order to cause a vaporization of the vaporizable material.

2. The vaporizer device of claim 1, wherein the controller is further configured to adjust, in response to detecting the change in the ambient pressure, a ramp rate at which a temperature of a heater of the vaporizer device is changed in order to achieve the setpoint temperature.

3. The vaporizer device of claim 2, wherein the controller applies a proportional- integral-derivative control to achieve the setpoint temperature, and wherein the controller adjusts the ramp rate by adjusting at least one of a proportional term, an integral term, or a derivative term of the proportional-integral-derivative control.

4. The vaporizer device of any of claims 2-3, wherein the ramp rate of the vaporizer device is adjusted such that the vaporizer device achieves the second temperature in a substantially same amount of time as the vaporizer device achieves the first temperature.

5. The vaporizer device of any of claims 1-4, wherein the setpoint temperature of the vaporizer device is adjusted to be equal to the second temperature.

6. The vaporizer device of any of claims 1-4, wherein the setpoint temperature of the vaporizer device is adjusted, based at least on a user preference for a strength of a vapor generated by the vaporization of the vaporizable material, to exceed the second temperature.

7. The vaporizer device of any of claims 1-6, wherein the setpoint temperature of the vaporizer device is further adjusted based at least on a type of the vaporizable material.

8. The vaporizer device of any of claims 1-7, wherein the adjustment of the setpoint temperature of the vaporizer device includes adjusting a preset setpoint temperature of the vaporizer device, wherein the preset setpoint temperature comprises a default setpoint temperature, a user defined setpoint temperature, and/or a previous setpoint temperature for the vaporizer device.

9. The vaporizer device of any of claims 1-8, wherein the controller is configured to respond to detecting the change in the ambient pressure by sending, to a user device coupled with the vaporizer device, a notification.

10. The vaporizer device of claim 9, wherein the notification indicates at least one of the change in the ambient pressure or the adjustment of the setpoint temperature of the vaporizer device.

11. The vaporizer device of any of claims 9-10, wherein the notification prompts a user to consent to the adjustment to the setpoint temperature of the vaporizer device, wherein the controller adjusts the setpoint temperature of the vaporizer device in response to the user consenting to the adjustment to the setpoint temperature of the vaporizer device.

12. The vaporizer device of any of claims 1-11, further comprising:
a vaporizer body, the vaporizer body including a cartridge receptacle configured to receive a cartridge containing the vaporizable material, the cartridge further including a heater configured to deliver heat to the vaporizable material contained in the cartridge, and the heat delivered to the vaporizable material causing the vaporization of the vaporizable material.

13. The vaporizer device of claim 12, wherein the controller is further configured to:
detect an insertion of the cartridge into the cartridge receptacle; and
respond to the insertion of the cartridge by at least activating the pressure sensor to measure the ambient pressure.

14. The vaporizer device of any of claims 1-13, further comprising:
a mouthpiece configured to enable a vapor to be drawn from the vaporizer device, the vapor being generated by the vaporization of the vaporizable material.

15. A method, comprising:
detecting, at a vaporizer device, a change in an ambient pressure from a first ambient pressure to a second ambient pressure, the first ambient pressure associated with a first temperature at which a vaporizable material undergoes a phase transition, and the second ambient pressure associated with a second temperature at which the vaporizable material undergoes the phase transition; and
in response to detecting the change in the ambient pressure, adjusting a setpoint temperature of the vaporizer device, the setpoint temperature being adjusted based at least on the second temperature at which the vaporizable material undergoes the phase transition at the second ambient pressure, and the vaporizer device operating at the setpoint temperature in order to cause a vaporization of the vaporizable material.

16. The method of claim 15, further comprising:
adjusting, in response to detecting the change in the ambient pressure, a ramp rate at which a temperature of a heater of the vaporizer device is changed in order to achieve the setpoint temperature.

17. The method of claim 16, further comprising:
applying a proportional-integral-derivative control to achieve the setpoint temperature, the adjusting of the ramp rate including adjusting at least one of a proportional term, an integral term, or a derivative term of the proportional-integral-derivative control.

18. The method of any of claims 16-17, wherein the ramp rate of the vaporizer device is adjusted such that the vaporizer device achieves the second temperature in a substantially same amount of time as the vaporizer device achieves the first temperature.

19. The method of any of claims 15-18, wherein the setpoint temperature of the vaporizer device is adjusted to be equal to the second temperature.

20. The method of any of claims 15-18, wherein the setpoint temperature of the vaporizer device is adjusted, based at least on a user preference for a strength of a vapor generated by the vaporization of the vaporizable material, to exceed the second temperature.

21. The method of any of claims 15-20, wherein the setpoint temperature of the vaporizer device is further adjusted based at least on a type of the vaporizable material.

22. The method of any of claims 15-21, wherein the adjusting of the setpoint temperature of the vaporizer device includes adjusting a preset setpoint temperature of the vaporizer device, wherein the preset setpoint temperature comprises a default setpoint temperature, a user defined setpoint temperature, and/or a previous setpoint temperature for the vaporizer device.

23. The method of any of claims 15-22, further comprising:
responding to detecting the change in the ambient pressure by at least sending, to a user device coupled with the vaporizer device, a notification.

24. The method of claim 23, wherein the notification indicates at least one of the change in the ambient pressure or the adjustment of the setpoint temperature of the vaporizer device.

25. The method of any of claims 23-24, wherein the notification prompts a user to consent to the adjusting of the setpoint temperature of the vaporizer device, wherein the setpoint temperature of the vaporizer device is adjusted in response to the user consenting to the adjusting of the setpoint temperature of the vaporizer device.

26. The method of any of claims 15-25, wherein the vaporizer device includes a vaporizer body, wherein the vaporizer body includes a cartridge receptacle configured to receive a cartridge containing the vaporizable material, wherein the cartridge further includes a heater configured to deliver heat to the vaporizable material contained in the cartridge, and wherein the heat delivered to the vaporizable material causes the vaporization of the vaporizable material.

27. The method of claim 26, further comprising:
detecting an insertion of the cartridge into the cartridge receptacle; and
responding to the insertion of the cartridge by at least activating a pressure sensor at the vaporizer device to measure the ambient pressure.

28. The method of any of claims 15-27, wherein the vaporizer device includes a mouthpiece configured to enable a vapor to be drawn from the vaporizer device, wherein the vapor is generated by the vaporization of the vaporizable material.

29. A non-transitory computer readable medium storing instructions, which when executed by at least one data processor, result in operations comprising:
detecting, at a vaporizer device, a change in an ambient pressure from a first ambient pressure to a second ambient pressure, the first ambient pressure associated with a first temperature at which a vaporizable material undergoes a phase transition, and the second ambient pressure associated with a second temperature at which the vaporizable material undergoes the phase transition; and
in response to detecting the change in the ambient pressure, adjusting a setpoint temperature of the vaporizer device, the setpoint temperature being adjusted based at least on the second temperature at which the vaporizable material undergoes the phase transition at the second ambient pressure, and the vaporizer device operating at the setpoint temperature in order to cause a vaporization of the vaporizable material.

30. An apparatus, comprising:
means for measuring an ambient pressure;
means for detecting a change in an ambient pressure from a first ambient pressure to a second ambient pressure, the first ambient pressure associated with a first temperature at which a vaporizable material undergoes a phase transition, and the second ambient pressure associated with a second temperature at which the vaporizable material undergoes the phase transition; and
means for responding to the change in the ambient pressure by adjusting a setpoint temperature of the apparatus, the setpoint temperature being adjusted based at least on the second temperature at which the vaporizable material undergoes the phase transition at the second ambient pressure, and the apparatus operating at the setpoint temperature in order to cause a vaporization of the vaporizable material.

31. The apparatus of claim 30, further comprising means for performing any of claims 16-28.
